(19) United States
(12) Patent Application Publication
(10) Pub. No.: US 2010/0160992 A1
(43) Pub. Date: Jun. 24, 2010

(54) IMPLANTABLE MEDICAL DEVICE, SYSTEM AND METHOD

(75) Inventor: Andreas Blomqvist, Spanga (SE)

Correspondence Address: SCHIFF HARDIN, LLP;PATENT DEPARTMENT, 233 S. Wacker Drive-Suite 6600, CHICAGO, IL, 60606-6473, US

(73) Assignee: St. Jude Medical AB

(21) Appl. No.: 12/600,542

(22) PCT Filed: May 28, 2007
(86) PCT No.: PCT/SE07/00511

§ 371 Date: Nov. 17, 2009

(51) Int. Cl. A61N 1/365

(52) U.S. Cl. 607/17

(57) ABSTRACT

In an implantable medical device and method for monitoring a lung deficiency in a patient, a housing containing a control circuit. Is implanted in a patient. The control circuit is configured to analyze one or more signals that represent the breathing of the patient. The control circuit is also configured to monitor a relationship between an expiratory phase and an inspiratory phase of the breathing cycle, or an analogous relationship, and to monitor a change in the lung deficiency by monitoring a change in this relationship.

### BACKGROUND OF THE INVENTION

1. Field of the Invention

The present invention relates to an implantable medical device, in particular for monitoring the pulmonary condition of a patient.

The invention also concerns an implantable medical system that includes such an implantable medical device.

The invention also relates to a method of monitoring a pulmonary condition.

2. Description of the Prior Art

United States Patent Application Publication No. 2005/0142070 describes an external respiratory therapy device that incorporates sensors that may be used to sense physiological conditions or parameters associated with pulmonary disease. The sensed conditions may be used to detect and/or to assess a presence of various types of pulmonary diseases. The assessment of the pulmonary disease may be utilized to control a drug therapy delivered to the patient to treat the pulmonary disease.

United States Patent Application Publication No. 2006/0195149 describes cardiac monitoring and/or stimulation methods and systems employing dyspnea measurement. An implantable cardiac device may sense transthoracic impedance and determine a patient activity level. The document describes that a system may include a lead system and a pulse generator that may incorporate one or more transthoracic impedance sensors that may be used to acquire the patient's respiration waveform, or other respiration-related information.

United States Patent Application Publication No. 2005/0043644 describes an approach for predicting disordered breathing that involves detecting one or more conditions associated with disordered breathing. The detected conditions are compared to disordered breathing prediction criteria. The document describes one method for detecting disordered breathing that involves monitoring a respiratory waveform output, for example, using a transthoracic impedance sensor. When the tidal volume of the patient's respiration, as indicated by the transthoracic impedance signal, falls below a hypopnea threshold, then a hypopnea event is declared.

United States Patent Application Publication No. 2006/0079802 describes a method and apparatus for detecting and monitoring obstructive sleep apnea. The apparatus includes an intracardiac impedance sensor to measure intracardiac impedance, a movement sensor to measure an amount of movement of a patient, and a controller operatively coupled to said intracardiac impedance sensor and said movement sensor, said controller being adapted to receive at least one of an intracardiac impedance and the amount of movement of the patient and to detect obstructive sleep apnea based upon said intracardiac impedance and said movement.

U.S. Pat. No. 6,949,075 describes that adventitious lung sounds indicative of lung congestion are detected using an implantable sensor. The sensor is adapted to be positioned adjacent to a pulmonary system and to output signals indicative of lung sounds in response to pulmonary system activity. A controller receives the signals and processes the signals to detect the presence of adventitious lung sounds. A respiratory cycle sensor operating in conjunction with the lung-sound sensor enables classification of an adventitious lung sound according to its time occurrence within the respiratory cycle. Posture sensing in conjunction with lung-sound sensing provides valuable additional information as to the severity of the lung congestion.

U.S. Pat. No. 5,683,427 describes a heart stimulator which has a pulse generator which periodically emits stimulation pulses, at least one electrode connectable to the pulse generator and to the heart for transmitting said pulses to the heart and a respiration monitor for monitoring the respiration of the pacemaker user. The heart stimulator adapts the energy to be delivered in a stimulation pulse in response to the information acquired by the respiration monitor which indicates the current stage in the user's respiration cycle.

U.S. Pat. No. 4,757,815 describes a heart pacemaker that has a pulse generator and circuitry for measuring a respiration signal of the user and a control unit for controlling the pulse generator by changing the pulse repetition rate dependent on the respiration signal. A heart action detector is provided for acquiring heart action signals and the respiration signal measuring circuitry includes detectors for measuring the amplitude fluctuations in the heart action signal and supplying those fluctuations to the control unit. It is described that the heart action signals are acquired by a heart pacemaker in a conventional manner, such as by means of a QRS detector. The amplitudes thereof are subject to fluctuations caused by the respiration cycle of the user of the heart pacemaker. The envelope for all such fluctuations corresponds to the respiration signal.

### SUMMARY OF THE INVENTION

For certain patients it is important to be able to monitor the status or progress of a lung deficiency, such as Chronic Obstructive Pulmonary Disease (COPD). It can be important to monitor such a disease or deficiency over a longer period, in order to monitor the long-term change of the lung deficiency. It may also be essential to detect sudden changes in the lung deficiency, in order to detect if the lung deficiency suddenly changes to the worse, in order to be able to take appropriate measures. The above cited documents give examples of how to monitor the respiration cycle or the like. However, the inventor of the present invention has realized that it is essential to find an improved implantable device and method for monitoring a lung deficiency, such as COPD.

An object of the present invention is to provide an implantable medical device with which it is possible to monitor a lung deficiency in a correct and efficient manner. A further object is to provide such a device which has a relatively simple construction. Still an object is to provide such a device which is able to detect quite sudden changes in the lung deficiency.

The above objects are achieved by an implantable medical device for monitoring a lung deficiency in a patient. The device has a housing and a control circuit within the housing. The control circuit is configured to analyze one or more signals which represent the breathing of said patient. The control circuit is configured to be able to monitor a relationship between an expiratory phase and an inspiratory phase of the breathing cycle, or an analogous relationship, and to monitor a change in said lung deficiency by monitoring a change in said relationship.

By studying said relationship it is thus possible to monitor a lung deficiency, such as COPD, in order to see how the lung deficiency progresses. For example, if the COPD becomes worse, then normally the expiratory phase of the breathing cycle becomes essentially longer, while the length of the inspiratory phase of the breathing cycle changes less. The inventor of the present invention has thus found that by monitoring this relationship, a very accurate indication of the progress of the lung deficiency is obtained.

A breathing cycle (BC) can be defined as an inspiratory phase (IP) followed by an expiratory phase (EP); i.e. concerning the duration of the breathing cycle BC=IP+EP. In case the patient in question were to pause the breathing during a short time interval between the expiration and the following inspiration (or between the inspiration and the expiration), then such a time interval may for example be said to belong to one of the phases or, alternatively, such an interval can be said to belong partly (e.g. 50%) to the expiratory phase and partly (e.g. 50%) to the inspiratory phase. If the patient for some reason were to pause the breathing during the inspiration (or during the expiration), then such a pause may be said to constitute part of the inspiratory phase (or the expiratory phase).

As used herein an “analogous relationship” means a relationship which includes basically the same information concerning the lung deficiency in question as the relationship between an expiratory phase and an inspiratory phase of the breathing cycle. The basis of the invention is thus to analyze the expiration in relation to the inspiration. However, since a breathing cycle is constituted by the inspiration phase and the expiration phase, a relationship, concerning for example time, between the expiration phase and the whole breathing cycle is to be considered as “analogous” to the relationship between the expiration and the inspiration (in fact, since BC=IP+EP, a relationship, concerning time, between the EP and BC can simply be recalculated into a relationship between EP and IP; similarly, a relationship between the IP and BC can also be recalculated into a relationship between EP and IP).

In an embodiment of the device according to the invention, the aforementioned relationship is the ratio between the duration of said expiratory phase and the duration of the inspiratory phase, or an analogous relationship. It has been found to be particularly advantageous and simple to monitor said ratio.

The relationship may thus simply be the duration of the expiration divided by the duration of the inspiration during a breathing cycle. However, also analogous relationships may be used, such as the duration of the expiration divided by the length of the whole breathing cycle, or the duration of the inspiration divided by the length of the whole breathing cycle or the duration of the inspiration divided by the duration of the expiration.)

In a further embodiment of the device according to the invention, the relationship is a relationship between the rate of change during the expiratory phase of a signal which represents the breathing of the patient and the rate of change during the inspiratory phase of the signal. The rate of change may simply be the derivative of the signal in question. If for example the lung deficiency becomes worse, the rate of change of the signal during the expiratory phase normally decreases more than the rate of change during the inspiratory phase. Consequently, the rates of change (or derivatives) may be used for forming the aforementioned relationship.

In a further embodiment of the device according to the invention, the device is configured to be connectable to at least one lead with at least one electrode and wherein the control circuit is configured to be able to derive, with the help of the at least one lead, the impedance over at least a portion of the patient's body, and the control circuit is configured to use the variation of this impedance as a signal representing the breathing of the patient. The variation of the impedance can thus be used as a signal representing the breathing. This is known from some of the documents cited above. However, it should be noted that the invention is not at all restricted to the use of impedance for detecting the breathing cycle. Any other suitable manner of detecting the breathing cycle can be used, such as a pressure sensor (for example positioned in the pericardium), the IEGM signal (see the above cited U.S. Pat. No. 4,757,815) or a microphone that detects the breathing, etc.

According to a further embodiment of the device according to the invention, the device has at least one memory, and the control circuit is configured to carry out a procedure that includes determining said relationship and storing a corresponding value in said memory. By storing such a value in the memory, it is possible to later retrieve information of the determined relationship, and thereby of the status of the lung deficiency.

In a further embodiment of the device according to the invention, the control circuit is configured to carry out the procedure at different occasions in time, in order to monitor if said relationship changes with time. Such occasions may for example occur each hour or once a day. This makes it possible to monitor how the lung deficiency changes over time. By carrying out the procedure quite often, it is possible to monitor quite sudden changes in the lung deficiency. By carrying out the procedure less frequently, it is possible to monitor the long-term change in the lung deficiency.

It should be noted that at each of these occasions, the relationship may, with advantage, be formed a number of times, and a representative value (for example the mean value of said plurality of relationships formed) can then be stored in the memory. For example, within one minute the relationship can be formed at least three times and the mean value can be stored in the memory. By forming such a representative value based on several measurements, a statistically more accurate value can be obtained.

According to a further embodiment of the device according to the invention, the device is configured to be able take into account, in addition to the relationship, also at least one further sensed property of the patient for evaluating said lung deficiency. The further property is preferably not directly related to the signal representing the breathing pattern of the patient. By taking some further sensed property into account, a more accurate result may be obtained.

According to a further embodiment of the device according to the invention, the at least one further property is one or more properties selected from the group that comprises the hematocrit value of the blood, the oxygen level in the blood, a sound effect received from a microphone. It has been noted that when the lung deficiency, such as COPD, becomes worse, then, over a long time, the hematocrit value tends to increase. This is the body's response in order to improve the oxygen transport in the blood. Similarly, the oxygen level in the blood tends to change when the lung deficiency changes. Furthermore, with the help of a microphone a sound effect, such as a wheezing sound, related to the breathing can be detected. Such further properties therefore include information concerning the lung deficiency in question. Consequently, such further properties can be used in combination with the above described relationship in order to monitor the lung deficiency.

According to a further embodiment of the device according to the invention, the device is provided with warning means, with which the patient in whom the device is implanted, or a medical practitioner, can be alerted. It is advantageous to warn the patient or the medical practitioner when necessary. The warning to the patient may for example be a vibration of the implanted device, which vibration is sensed by the patient. The medical practitioner can for example be alerted if the device emits a signal that is detected by a receiver outside of the patient, such that this receiver can forward a message in order to alert the medical practitioner.

According to a further embodiment of the device according to the invention, the control circuit is configured to trigger the warning means to emit an alert signal if the relationship, and possible further sensed property or properties of the patient, fulfils a predetermined criteria related to the severity of said lung deficiency. It is thus advantageous to monitor the lung deficiency and to emit an alert signal if the lung deficiency in question is severe. The patient, or the medical practitioner, can then take appropriate measures.

According to a further embodiment of the device according to the invention, the control circuit is configured to trigger the warning means if the calculated severity of said lung deficiency, based on said relationship, and possible further sensed property or properties of the patient, has changed to the worse more than a predetermined amount such that an acute attack caused by the lung deficiency is likely to be imminent.

According to this embodiment, it is thus possible to warn the patient or the medical practitioner if an acute attack is imminent. A lung deficiency, such as COPD, may become worse to such an extent that the patient suffers an acute attack, which may be similar to the attacks of asthma patients. Such an attack may have severe consequences. It is therefore a particular advantage of the present invention that it is possible to predict such an attack with high accuracy. By warning the patient or the medical practitioner when such an attack is imminent, appropriate measures may be taken, such that a full-blown attack can be avoided.

According to a further embodiment of the device according to the invention, the device is configured to also be able to deliver pacing pulses to the heart of the patient in order to function as a cardiac pacemaker. It is advantageous for the device according to the invention to also be able to function as a cardiac pacemaker. Such a cardiac pacemaker may have functions that can be used also for monitoring the respiratory cycle. Consequently, such a device can be constructed to also be used for monitoring a lung deficiency as explained above. It should be noted that the device may of course also have further functions, for example in order to function as a defibrillator.

A further object of the invention is to provide an advantageous implantable medical system including a medical device according to the invention.

This object is achieved by an implantable medical system including an implantable medical device as described above and at least one lead connected to the device, wherein said lead comprises at least one electrode suitable for delivering pacing pulses and/or for being used when measuring, by means of the device, the impedance over at least a portion of the patients body.

With such a system, the advantages described above in connection with the device according to the invention can be obtained.

Another object of the invention is to provide a method with which it is possible to monitor a lung deficiency in a correct and efficient manner. A further object is to provide such a method which is relatively simple. Another object is to provide such a method with which it is possible to detect quite sudden changes in the lung deficiency.

These objects are achieved by a method of monitoring a lung deficiency in a patient with the help of an implantable medical device, wherein the method includes the following steps:

implant the device in the patient,

analyze one or more signals which represent the breathing of said patient,

monitor a relationship between an expiratory phase and an inspiratory phase of the breathing cycle, or an analogous relationship,

monitor a change in said lung deficiency by monitoring a change in the relationship.

The method according to the invention has similar advantages to those described above in connection with the device according to the invention.

### BRIEF DESCRIPTION OF THE DRAWINGS

FIG. 1 schematically illustrates an embodiment of an implantable medical device according to the invention, connected to leads with electrodes/sensors arranged in or outside a heart.

FIG. 2 schematically illustrates an embodiment of a control circuit of the implantable medical device according to the invention.

FIG. 3 schematically illustrates respiration detected in accordance with the present invention.

FIG. 4 schematically illustrates detected respiration similar to that of FIG. 3, but within a different time period.

FIG. 5 schematically illustrates an embodiment of the method according to the present invention.

### DESCRIPTION OF THE PREFERRED EMBODIMENTS

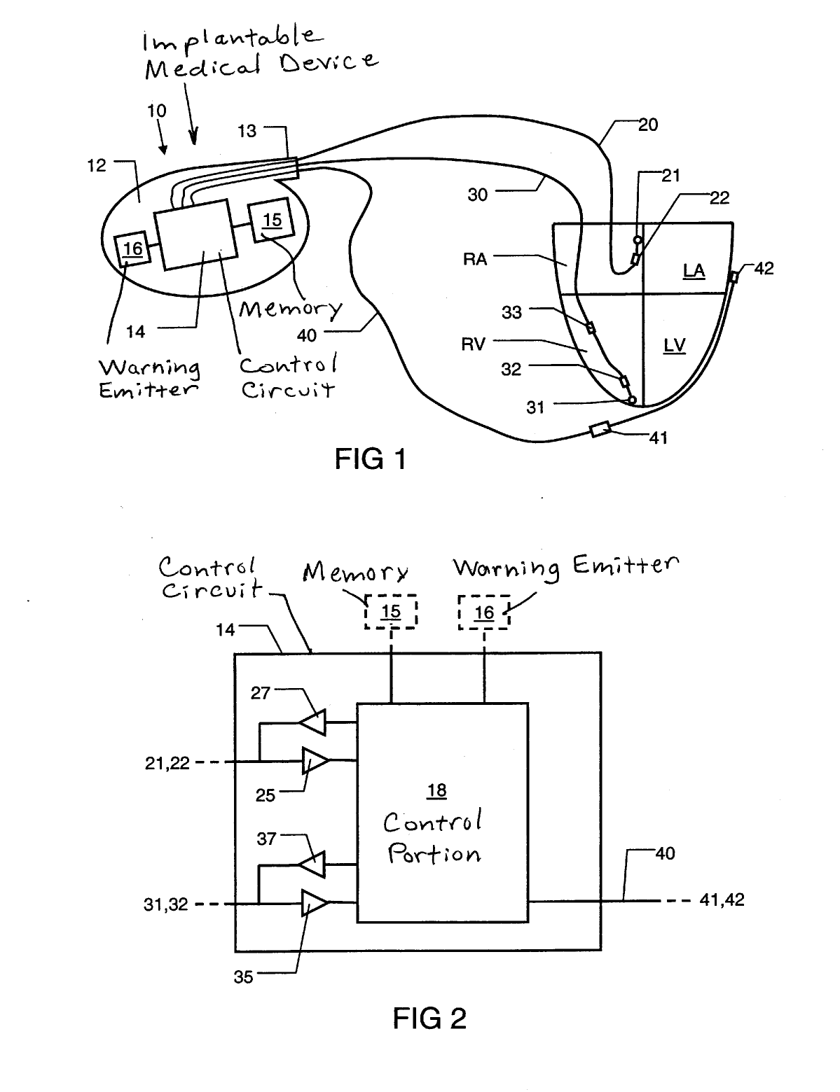

FIG. 1 shows schematically an embodiment of an implantable medical device 10 according to the invention for monitoring a lung deficiency in a patient. According to the shown embodiment, the device 10 also functions as a heart stimulating device. The device 10 has a housing 12. The device 10 has a connector portion 13. Via the connector portion 13, the device 10 can be connected to different leads. In FIG. 1 the device 10 is connected to three leads 20, 30 and 40.

FIG. 1 also schematically shows a heart with a right atrium RA, a left atrium LA, a right ventricle RV and a left ventricle LV.

The lead 20 includes a pacing and sensing electrode 21, 22.

Similarly to the lead 20, the lead 30 includes a pacing and sensing electrode 31, 32. The lead 30 has a further sensor 33, which may be, for example, a sensor for sensing the hematocrit value of the blood or a sensor for sensing the oxygen level in the blood. Such sensors are known in the art, see, for example, PCT application PCT/SE2007/000165 and WO 00/56397.

A third lead 40 is provided with an electrode 41 and a sensor 42. The electrode 41 can for example, be used in combination with some further electrode (or the housing 12) for measuring transthoracic impedance. The sensor 42 may be a microphone for detecting respiratory sounds. This microphone 42 may be positioned, for example, in the pericardium. The microphone 42 can be used to detect sounds caused by the breathing of the patient, for example a wheezing sound that indicates the lung deficiency in question.

In the shown example, the electrodes 21, 22, 31, 32 are bipolar electrodes with a tip portion 21, 31 and a ring portion 22, 32. However, it is also possible to use unipolar electrodes. This is known to those skilled in the art.

The device 10 has a control circuit 14, which will be described further below. The device 10 also has at least one memory 15 connected to the control circuit 14. Furthermore, the device 10 may be provided with warning emitter 16 with which the patient in whom the device 10 is implanted, or a medical practitioner, can be alerted. The warning emitter 16 may be, for example, a vibrator that causes the device 10 to vibrate in order to alert the patient. The warning emitter 16 may also be a transmitter that transmits a signal to a receiver outside of the patient, which receiver is used to alert a medical practitioner.

The device 10 together with the leads 20, 30, 40 and the pacing/sensing electrodes 21, 22, 31, 32 and the electrode/sensors 33, 41, 42 constitute a heart stimulating system according to the invention. This system can be implanted in a patient.

It can be noted that FIG. 1 shows a device 10 which can electrically pace and sense both an atrium and a ventricle. However, also other alternatives are possible. The device 10 can of course also constitute a monitoring device 10, which is not designed to pace the heart.

In FIG. 1, the electrode 21, 22 constitutes an atrial sensing and/or pacing electrode 21, 22 which is positioned in an atrium of the heart, according to this embodiment the right atrium RA, in order to enable sensing and/or pacing of this atrium RA.

The electrode 31, 32 constitutes a ventricular sensing and pacing electrode 31, 32, which in this embodiment is positioned in the right ventricle RV. This sensing and pacing electrode 31, 32 is adapted to enable sensing and pacing of the right ventricle RV.

It is also possible that the device 10 is connected to further leads and/or further sensing/pacing electrodes, for example electrodes positioned in order to sense and/or pace the left ventricle LV and/or the left atrium LA and electrodes designed to enable defibrillation.

FIG. 2 shows schematically the control circuit 14 in some more detail. The control circuit 14 includes a control portion 18 connected to the memory 15 and to the warning means 16.

The control circuit 14 includes an atrial sensing and/or pacing circuit 25, 27. In this embodiment, this circuit 25, 27 includes a sensing circuit 25 and a pacing circuit 27. The atrial sensing and/or pacing circuit 25, 27 communicates with the atrial sensing and/or pacing electrode 21, 22 via the lead 20. The atrial sensing and/or pacing circuit 25, 27 is thus adapted to sense and/or pace an atrium, in this case the right atrium RA.

The control circuit 14 also includes a ventricular sensing circuit 35 and a ventricular pacing circuit 37. These circuits 35, 37 communicate with the ventricular sensing and pacing electrode 31, 32 via the lead 30. The circuits 35, 37 are thus adapted to sense and pace a ventricle, in this case the right ventricle RV.

As indicated in FIG. 2, the electrode 41 and sensor 42 are also connected to the control circuit 14, via the lead 40.

The control circuit 14 is arranged to be able to operate in time cycles corresponding to heart cycles. Such an operation is normal for an implantable heart stimulating device. The time cycles are determined by preset timer intervals which also may depend on detected signals.

The control circuit 14 is also configured to be able to analyse one or more signals which represent the breathing of the patient. This can for example be done by measuring the impedance over at least a portion of the patient's body. For example a transthoracic impedance may be measured, as indicated in some of the above-cited documents. The impedance may for example be measured between the housing 12 of the device 10 and an electrode surface, such as the ring portion 32. However, many other alternatives are possible. It is for example possible to measure the impedance between the electrode 41 and another electrode 21, 22, 31, 32 or the housing 12.

The measured impedance varies with the breathing of the patient.

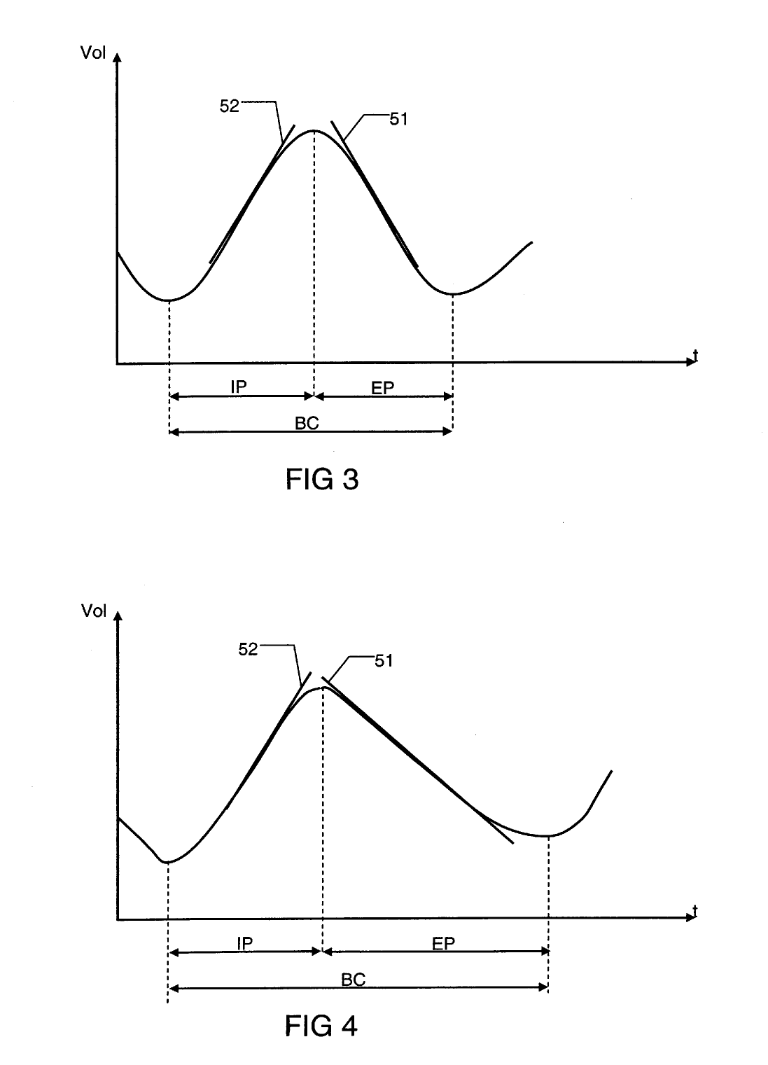

FIG. 3 illustrates the detected respiration. Vol represents the lung volume and t the time. The variation of the lung volume may thus for example be derived through the mentioned impedance measurement. In FIG. 3, BC represents a breathing cycle including an inspiratory phase IP and an expiratory phase EP.

The control circuit 14 is configured to monitor a relationship between the expiratory phase EP and the inspiratory phase IP of the breathing cycle, or an analogous relationship as explained above. The control circuit 14 is configured to monitor a change in the lung deficiency by monitoring a change in the relationship.

The aforementioned relationship may be the duration of the expiratory phase EP divided by the duration of the inspiratory phase IP, or an analogous relationship. When the lung deficiency, such as COPD, becomes worse, EP becomes longer while IP does not change as much as EP. Consequently, the ratio EP/IP increases when the COPD becomes worse.

FIG. 4 illustrates the respiration in the same manner as in FIG. 3. However, it can be seen that in FIG. 4 EP has become longer compared to in FIG. 3. Consequently, in FIG. 4 the ratio EP/IP is higher than the ratio EP/IP in FIG. 3. FIG. 4 may represent the respiration of a patient at a later occasion, than the occasion illustrated in FIG. 3. This indicates that the lung deficiency has become worse.

FIGS. 3 and 4 also indicate the rate of change (or derivative) 51 of the breathing signal during the EP as well as the rate of change (or derivative) 52 of the breathing signal during the IP. Also the relationship between the rate of change 51 during the EP and the rate of change 52 during the IP may be used as an indication of the lung deficiency. It can be seen that the absolute value of the derivative 51 is lower in FIG. 4 than in FIG. 3. The rates of change during the EP and the IP can be determined in any suitable manner. These rates of change may for example be the maximum absolute value of the derivative during the EP and IP, respectively.

The control circuit 14 is configured to carry out the procedure of determining the mentioned relationship at different occasions in time and to store a corresponding value in the memory 15. In this manner, the control circuit 14 may monitor how the relationship, and thereby the lung deficiency, changes with time.

The control circuit 14 may also be configured to take further sensed properties of the patient into account for evaluating the lung deficiency. Such properties may for example be the hematocrit value or the oxygen level of the blood, derived for example with the help of the sensor 33, or a sound effect received from the microphone 42, which represents a breathing sound of the patient.

According to this embodiment, the control circuit 14 is configured to trigger the warning means 16 to emit an alert signal if the mentioned relationship (for example the duration of EP divided by the duration of IP) has changed to the worse. The control circuit 14 may for example monitor this relationship and if the relationship changes it is possible to monitor this change and to emit an alert signal if the change in the mentioned relationship indicates that an acute attack caused by the lung deficiency is likely to be imminent. The decision whether to trigger the warning means 16 may also depend on further sensed properties, for example those described above, in order to increase the redundancy and in order to be able to determine the state of the lung deficiency with higher accuracy.

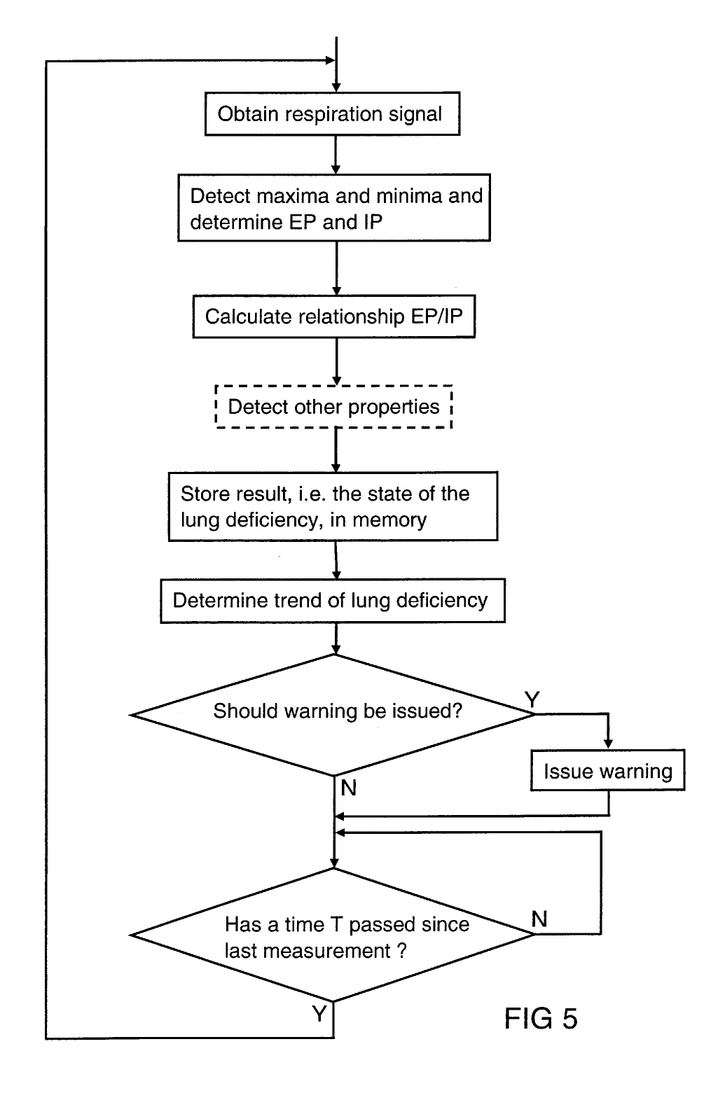

FIG. 5 illustrates schematically a method according to the invention. FIG. 5 can also be seen as an illustration of the operation of the device 10 according to the invention.

First (which is not shown in FIG. 5), the device 10 with the leads 20, 30, 40 and electrodes and sensors 21, 22, 31, 32, 33, 41, 42 is implanted in a patient. Then, if necessary, the device may be calibrated to the actual patient by suitable programming steps performed by the medical practitioner. Thereafter, as shown in FIG. 5, a respiration signal (for example of the kind shown in FIG. 3 and FIG. 4) is obtained. Maxima and minima in this respiration signal is detected in order to determine the expiratory phase EP and the inspiratory phase IP of a breathing cycle. At the next step, the mentioned relationship, for example, EP/IP is calculated. Also other properties may be detected. Such properties may be, for example, the hematocrit value, the oxygen value or a lung sound as explained above.

The determined relationship and possibly also other detected properties are stored in the memory 15. The state of the lung deficiency is thus stored in the memory 15. The whole operation explained so far and shown in FIG. 5 is repeated at regular intervals, for example once an hour or once a day. However, the procedure can also be performed more frequently.

For example, if the lung deficiency is severe, or has changed to the worse, then the procedure may be repeated much more frequently, for example every fifth minute.

As shown in FIG. 5, the trend of the lung deficiency is determined from the stored information. This means that it is monitored whether the mentioned relationship (and possible further detected properties) has changed such that the lung deficiency has become worse (or possibly better).

At the next step it is decided whether the patient, or the medical practitioner, should be alerted with a warning. If the trend of the lung deficiency is such that the lung deficiency has become worse such that an acute attack caused by the lung deficiency is likely to be imminent, then it is decided that the patient, or the medical practitioner, should be alerted with a warning. If this is not the case, then the procedure continues with regular intervals.

According to the method, the mentioned relationship, with which the lung deficiency can be determined, is thus monitored and if the lung deficiency becomes worse it is possible to issue a warning. However, it should be noted that the possibility of issuing a warning is an optional feature. Instead, it is of course possible to only store the monitored indications of the lung deficiency in the memory 15 such that a medical practitioner, for example with the help of so-called telemetry, may obtain information of the progress of the lung deficiency.

As mentioned above, the relationship may be another relationship than the mentioned relationship between EP and IP. For example, the monitored relationship may comprise a relationship between the rate of change during the expiratory phase of a signal which represents the breathing of said patient and rate of change during the inspiratory phase of the signal.

As also mentioned above, the method may also include the delivery of pacing pulses to the heart of the patient in order to pace the heart. Furthermore, the method, and the device, may be provided with other means which are known in connection with implantable heart monitoring and stimulating devices.

Although modifications and changes may be suggested by those skilled in the art, it is the intention of the inventor to embody within the patent warranted hereon all changes and modifications as reasonably and properly come within the scope of his or her contribution to the art.

I claim as my invention:

1-24. (canceled)

25. An implantable medical device comprising:
a housing adapted for implantable in al living subject;
a control unit contained within said housing, said control unit being supplied with at least one signal representing respiration, comprising a plurality of breathing cycles, of the living subject; and
said control circuit being configured to monitor a relationship, as represented in said at least one signal, between an expiratory phase and an inspiratory phase of the breathing cycle, and to monitor a change in said relationship and to provide a control circuit output representing a change in lung deficiency of the living subject dependent on said change in said relationship.

26. An implantable medical device as claimed in claim 25 wherein said control circuit is configured to monitor, as said relationship, a ratio between a duration of the expiratory phase and a duration of the inspiratory phase.

27. An implantable medical device as claimed in claim 25 wherein said control circuit is configured to monitor, as said relationship, a relationship between a rate of change of said signal during said inspiratory phase at a rate of change of said signal during said inspiratory phase.

28. An implantable medical device as claimed in claim 25 comprising a memory accessible by said control circuit, and wherein said control circuit is configured to implement a procedure that results in said control circuit output and to store said control circuit output in said memory.

29. An implantable medical device as claimed in claim 28 wherein said control circuit is configured to implement said procedure at different occasions and to determine whether said relationship changes with time.

30. An implantable medical device as claimed in claim 25 wherein said control circuit receives a further signal representing a sensed physiological property of the patient, and wherein said control circuit is configured to emit said control circuit output dependent on said change in said relationship and on said further signal.

31. An implantable medical device as claimed in claim 30 comprising a warning signal emitter that emits a humanly perceptible signal in response to said control circuit output.

32. An implantable medical device as claimed in claim 31 wherein said control circuit is configured to determine a severity of said lung sufficiency from said relationship and to emit said control circuit output dependent on said severity, and wherein said warning emitter is configured to emit said humanly perceptible warning dependent on the severity of said lung deficiency represented by said control circuit output.

33. An implantable medical device as claimed in claim 32 wherein said warning emitter is configured to emit said warning signal if said severity of said lung deficiency represented by said control circuit output changes by more than a predetermined amount.

34. An implantable medical device as claimed in claim 25 comprising a pacing pulse generator adapted to deliver cardiac pacing pulses to said patient according to a pacing regimen controlled by said control circuit, and wherein said control circuit is configured to modify said pacing regimen dependent on said relationship.

35. An implantable medical system comprising:
a housing configured for implantation in a patient;
at least one medical lead adapted for implantation in the patient;
a control circuit in said housing connected to said at least one medical lead to receive an input signal therefrom representing breathing, in a breathing cycle, of said patient;
a therapy administration circuit that generates a therapy, according to a therapy regimen controlled by said control circuit, said therapy generating circuit being connected to said medical lead and delivering said therapy via said medical lead;
said control circuit being configured to monitor a relationship, represented by said input signal, between an inspiratory phase and an expiratory phase of the breathing cycle to monitor a change in lung deficiency by monitoring a change in said relationship, and to modify said therapy regiment dependent on said relationship.

36. An implantable medical device as claimed in claim 35 wherein said at least one medical lead is configured to detect and provide an impedance signal to said control circuit, as said input signal.

37. An implantable medical system as claimed in claim 35 wherein said therapy generating circuit is a pacing pulse generator that emits cardiac pacing pulses as said therapy.

38. An implantable medical system as claimed claim 36 comprising a sensor configured for implantation in said patient that emits a sensor signal representing a physiological property of the patient, said sensor being connected to said control circuit and said control circuit being configured to modify said therapy regimen dependent on said sensor signal and on said relationship.

39. An implantable medical system as claimed in claim 38 wherein said sensor is selected from the group consisting of a sensor allowing a hematocrit value of blood of the patient to be determined from said sensor signal, a sensor that measures an oxygen level in the blood of the patient, and a microphone that provides a signal representing a sound that occurs in the patient.

40. An implantable medical system as claimed in claim 35 comprising a warning signal emitter connected to said control circuit that emits a humanly perceptible warning signal dependent on said relationship.

41. A method for monitoring lung deficiency in a patient, comprising the steps of:
implanting a device within the patient;
in said device, automatically analyzing at least one signal representing breathing, in a breathing cycle, of the patient;
in said device, automatically monitoring a relationship between an expiratory phase and an inspiratory phase of said breathing cycle; and
in said device, monitoring a change of said relationship and emitting a signal representing a change in lung deficiency of the patient dependent on said change in said relationship.

42. An implantable medical system as claimed in claim 25 wherein said control circuit is configured to monitor, as said relationship, a ratio between a duration of the expiratory phase and a duration of the inspiratory phase.

43. An implantable medical system as claimed in claim 25 wherein said control circuit is configured to monitor, as said relationship, a relationship between a rate of change of said signal during said inspiratory phase at a rate of change of said signal during said inspiratory phase.

44. An implantable medical system as claimed in claim 25 comprising a memory accessible by said control circuit, and wherein said control circuit is configured to implement a procedure that results in said control circuit output and to store said control circuit output in said memory.

45. An implantable medical system as claimed in claim 28 wherein said control circuit is configured to implement said procedure at different occasions and to determine whether said relationship changes with time.

46. An implantable medical system as claimed in claim 25 wherein said control circuit receives a further signal representing a sensed physiological property of the patient, and wherein said control circuit is configured to emit said control circuit output dependent on said change in said relationship and on said further signal.

47. An implantable medical system as claimed in claim 30 comprising a warning signal emitter that emits a humanly perceptible signal in response to said control circuit output.

48. An implantable medical system as claimed in claim 31 wherein said control circuit is configured to determine a severity of said lung sufficiency from said relationship and to emit said control circuit output dependent on said severity, and wherein said warning emitter is configured to emit said humanly perceptible warning dependent on the severity of said lung deficiency represented by said control circuit output.

49. An implantable medical system as claimed in claim 32 wherein said warning emitter is configured to emit said warning signal if said severity of said lung deficiency represented by said control circuit output changes by more than a predetermined amount.

50. An implantable medical system as claimed in claim 25 comprising a pacing pulse generator adapted to deliver cardiac pacing pulses to said patient according to a pacing regimen controlled by said control circuit, and wherein said control circuit is configured to modify said pacing regimen dependent on said relationship.

51. An implantable medical system as claimed in claim 35 wherein said at least one medical lead is configured to detect and provide an impedance signal to said control circuit, as said input signal.

52. An implantable medical system as claimed in claim 38 wherein said sensor is selected from the group consisting of a sensor allowing a hematocrit value of blood of the patient to be determined from said sensor signal, a sensor that measures an oxygen level in the blood of the patient, and a microphone that provides a signal representing a sound that occurs in the patient.
